(19) United States
(12) Patent Application Publication
(10) Pub. No.: US 2016/0106574 A1
(43) Pub. Date: Apr. 21, 2016

(54) Thermal Body Suit

(71) Applicant: Shawn L. Stewart, Newport News, VA (US)

(72) Inventor: Shawn L. Stewart, Newport News, VA (US)

(21) Appl. No.: 14/696,779

(22) Filed: Apr. 27, 2015

Related U.S. Application Data
(60) Provisional application No. 62/065,796, filed on Oct. 20, 2014.

(51) Int. Cl. A61F 7/00

(52) U.S. Cl. CPC A61F 7/007; A61F 2007/0094

(57) ABSTRACT

A thermal body suit that provides heat therapy is provided. The thermal body suit includes a unitary body suit having an outer layer, an inner layer, and an insulation layer disposed between the outer layer and the inner layer. The insulation layer includes a plurality of far infrared (FIR) coils disposed therein. The plurality of FIR coils are sandwiched between the outer layer and the inner layer. A controller is provided for controlling the heat output of the plurality of the FIR coils. The controller includes an input device to facilitate entry of a desired temperature. A universal serial bus port is disposed on the outer layer of the unitary body suit. The universal serial bus port is in electrical communication with the FIR coils to provide power to the coils.

### CROSS REFERENCE TO RELATED APPLICATION

This application claims the benefit of U.S. Provisional Application No. 62/065,796 filed on Oct. 20, 2014. The above identified patent application is herein incorporated by reference in its entirety to provide continuity of disclosure.

### FIELD OF THE INVENTION

The invention generally relates to a body suit for providing heat therapy to a user. More specifically, the present invention relates to a form-fitting unitary body suit having an outer layer, an insulation layer, and an inner layer. A plurality of FIR coils are disposed in the insulation layer, sandwiched between the outer and inner layer. The FIR coils provide the desired level of heat to a user to help relieve pain from arthritis and fibromyalgia, thereby helping to assist in healthy blood flow.

### BACKGROUND OF THE INVENTION

Many people suffer from years of chronic, nagging pain due to joint, bone, and muscle related stress, yet cannot afford surgery which may address the fundamental cause of the pain. Arthritis, fibromyalgia, and frequent stress on bones, joints, and muscles from sports-related activities, contribute to the issues people suffer on a day-to-day basis, and their quality of life diminishes. These musculoskeletal disorders are characterized by widespread pain, fatigue, sleep, memory and mood issues. Many people who have fibromyalgia also have tension headaches, temporomandibular joint (TMJ) disorders, irritable bowel syndrome, anxiety and depression. While there is no cure for fibromyalgia or arthritis, a variety of medications can help control symptoms. Exercise, relaxation and stress-reduction measures also may help.

Many people try alternative therapies to help with the musculoskeletal disorders. These include acupuncture, hypnosis, physical therapy, frequency modulation, brainwave optimization, chiropractic, vitamins and supplements, guided meditation, herbal packs and a host of other therapies. Many people are able to reduce their symptoms with a combination of exercise, medication, physical therapy, relaxation and heat therapy, especially heat therapy and Infrared heat therapy. By promoting vasodilation and expanding the blood vessels, heat therapy increases blood flow. Increased circulation brings more oxygen and nutrients to the affected areas and accelerates removal of waste products. Increased metabolic rate accelerates chemical reactions involved in tissue healing and increased oxygen-hemoglobin dissociation increases oxygen delivery to the tissues.

In addition to rushing fresh blood to the damaged tissue, heat therapy helps to flush toxins and other wastes away from the area. Therapeutic heating reduces pain by activating thermo-receptors to block the perception of pain and by promoting vasodilatation in relatively ischemic tissues. Therapeutic heating contributes to increasing range of motion by increasing soft tissue extensibility. This leads many people to using treatments such as heating pads, wraps, and warm baths to relieve pain. These remedies, however, do not provide total body relief that is continuous for extended periods of time.

Some devices in the prior art have been disclosed that relate to thermal body suits, such as U.S. Pat. No. 6,927,316, U.S. Pat. No. 4,308,622, and U.S. Pat. No. 6,228,106. Other devices relate to specific articles of clothing that provide therapeutic heating to certain areas of the body, such as U.S. Pat. No. 6,550,471 and U.S. Pat. No. 8,133,264. These prior art devices have several known drawbacks. Such devices fail to provide a dual layered unitary body suit with integrated FIR coils. The prior art devices fail to provide a body suit with a built in FIR coils that provide therapeutic heating to the entire body of a user, as is provided by the present invention.

In light of these devices disclosed in the prior art, it is submitted that the present invention substantially diverges from the prior art and consequently it is clear that there is a need in the art for an improvement to existing thermal body suits. In this regard the instant invention substantially fulfills these needs.

### SUMMARY OF THE INVENTION

In view of the foregoing disadvantages inherent in the known types of thermal body suits now present in the prior art, the present invention provides a new and improved thermal body suits wherein the same can be utilized for providing convenience for the user when treating musculoskeletal disorders such as fibromyalgia, arthritis, and osteoporosis, as well as joint and muscle pain.

It is therefore an object of the present invention to provide a new and improved thermal body suit that has all of the advantages of the prior art and none of the disadvantages.

It is one object of the present invention to provide a thermal body suit that provides heat therapy which includes a unitary body suit. The unitary body suit includes an outer layer and an inner layer. An insulation layer is disposed between the outer layer and the inner layer and retains heat within the unitary body suit.

It is another object of the present invention to provide a thermal body suit that includes a plurality of FIR coils disposed in the insulation layer. The plurality of FIR coils are sandwiched between the outer layer and the inner layer to provide a compact and form-fitting design for the unitary body suit.

It is yet another object of the present invention to provide a thermal body suit that includes a universal serial bus port disposed on the outer layer of the unitary body suit. The universal serial bus port is in electrical communication with the FIR infrared coils and provides power to the coils.

Yet another object of the present invention is to provide a thermal body suit that includes a controller adapted to control the heat output of the plurality of FIR coils. The controller includes an input device to facilitate entry of a desired temperature by a user.

It is still yet another object of the present invention to provide a thermal body suit that further includes a fastener disposed on the body suit for opening the. The fastener can be a hook and loop attachment or a zipper. The fastener is disposed a side of the thermal body suit and allows a user to wear and remove the body suit.

It is yet a further object of the present invention to provide a thermal body suit that includes a remote control to set the temperature and time of application of the thermal body suit. The remote control also displays the temperature of the unitary body suit and the user via a temperature sensor.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the thermal body suit according to one embodiment of the present invention.

FIG. 2 shows an exploded view of the thermal body suit according to one embodiment of the present invention.

FIG. 3 shows a close up view of the thermal body suit with a universal serial bus port according to one embodiment of the present invention.

### DETAILED DESCRIPTION OF THE INVENTION

Reference is made herein to the attached drawings. Like reference numerals are used throughout the drawings to depict like or similar elements of the thermal body suit. For the purposes of presenting a brief and clear description of the present invention, the preferred embodiment will be discussed as used for providing heat to a user. The figures are intended for representative purposes only and should not be considered to be limiting in any respect.

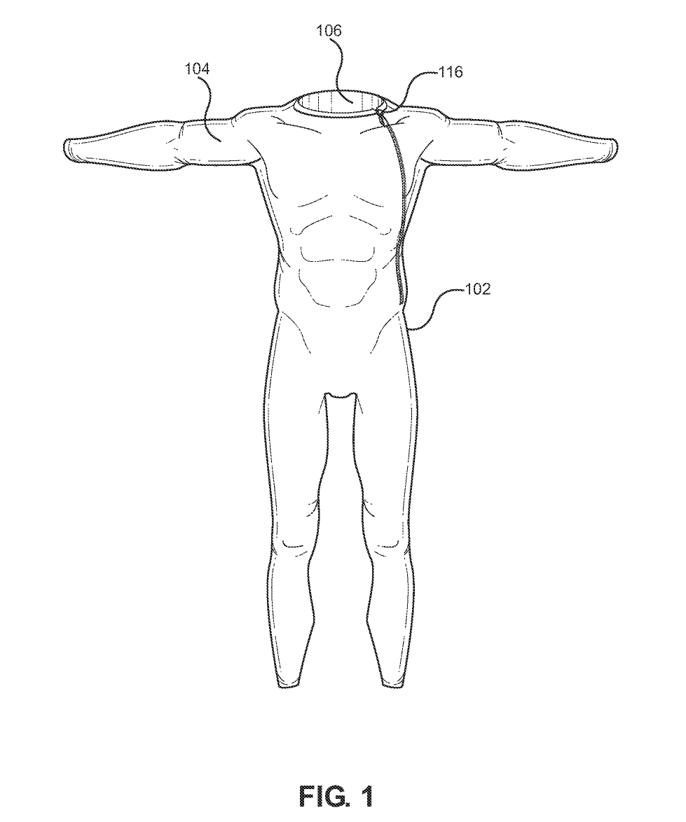

Referring now to FIG. 1, there is shown a perspective view of the thermal body suit according to one embodiment of the present invention. The thermal body suit includes a unitary body suit 102 that provides heat therapy. The unitary body suit 102 includes an outer layer 104 and an inner layer 106. An insulation layer is disposed between the outer layer 104 and the inner layer 106. The unitary body suit 102 may be fabricated from a foamed neoprene, providing thermal insulation, abrasion resistance and buoyancy.

According to one embodiment of the present invention, the unitary body suit 102 is configured to be worn on the body of a user. The unitary body suit 102 includes a torso portion having a neck opening, a pair of sleeves, and a pair of leg portions. The pair of sleeves extends from the torso portion. The sleeves include a shoulder end coupled to the torso portion, a wrist end opposite the shoulder end, a forearm portion closer to the wrist end than the shoulder end, and an upper arm portion closer to the shoulder end than the wrist end. Similarly, the leg portions extend from the torso portion and substantially covers a user's thighs and calf and terminates at an ankle region. The unitary body suit 102 can be provided in a variety of sizes to snugly fit a user. The unitary body suit 102 may further include straps at the wrist ends and the leg ends to provide a snug fit on the user.

The unitary body suit 102 includes a fastener 116 disposed on a side of the unitary body suit 102. The fastener 116 allows a user to easily wear or remove the therapeutic garment. The fastener 116 may be a zipper extending from the top of the chest area to the bottom of the leg. In an alternate embodiment, the fastener may be a plurality of hook and loop attachments disposed along a length of the outer layer 104. The unitary body suit 102 is configured to cover the arms, legs, and torso of a user.

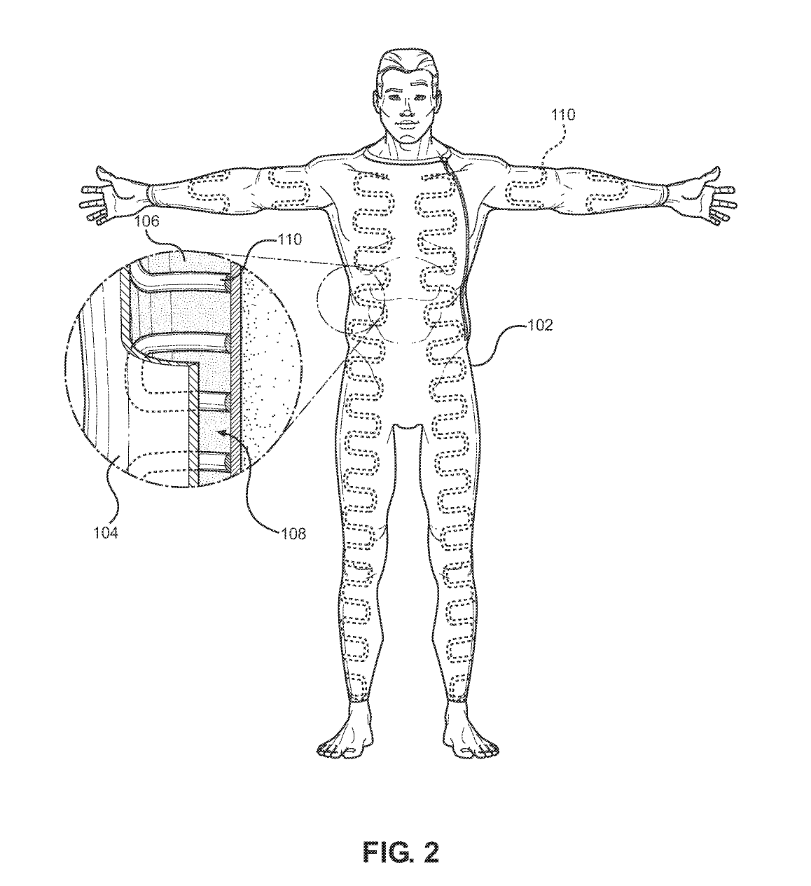

Referring now to FIG. 2, there is shown an exploded view of the thermal body suit according to one embodiment of the present invention. The unitary body suit 102 includes an outer layer 104 and an inner layer 106 with an insulation layer 108 interdisposed between the outer layer 104 and the inner layer 106. In one embodiment, the insulation layer 108 is a space provided between the outer layer 104 and the inner layer 106 that includes a gas, preferably air.

A plurality of FIR coils 110 are disposed in the insulation layer 108. The coils 110 are disposed throughout the suit so that there are coils on the user's torso, arms, and legs. The FIR infrared coils 110 are sandwiched between the outer layer 104 and the inner layer 106. The FIR infrared coils 110 produces FAR infrared rays that provide heat. FAR infrared rays are waves of energy that are invisible to the naked eye, capable of penetrating deep into the human body, where they elevate the body's surface temperature and activate major bodily functions. FIR can penetrate deeply into the tissues, generating the sensation of warmth as they do so. The FIR produces a resonance in the body, thus generating this feeling of gentle warmth and repair. A power source, such as a rechargeable battery unit, is in electrical communication with the FIR infrared coils via a USB port. In one embodiment, the battery unit is an external unit that is connected to the body suit via the USB port. In another embodiment, the rechargeable battery unit is incorporated into the body suit and includes a battery storage to hold charge for several hours.

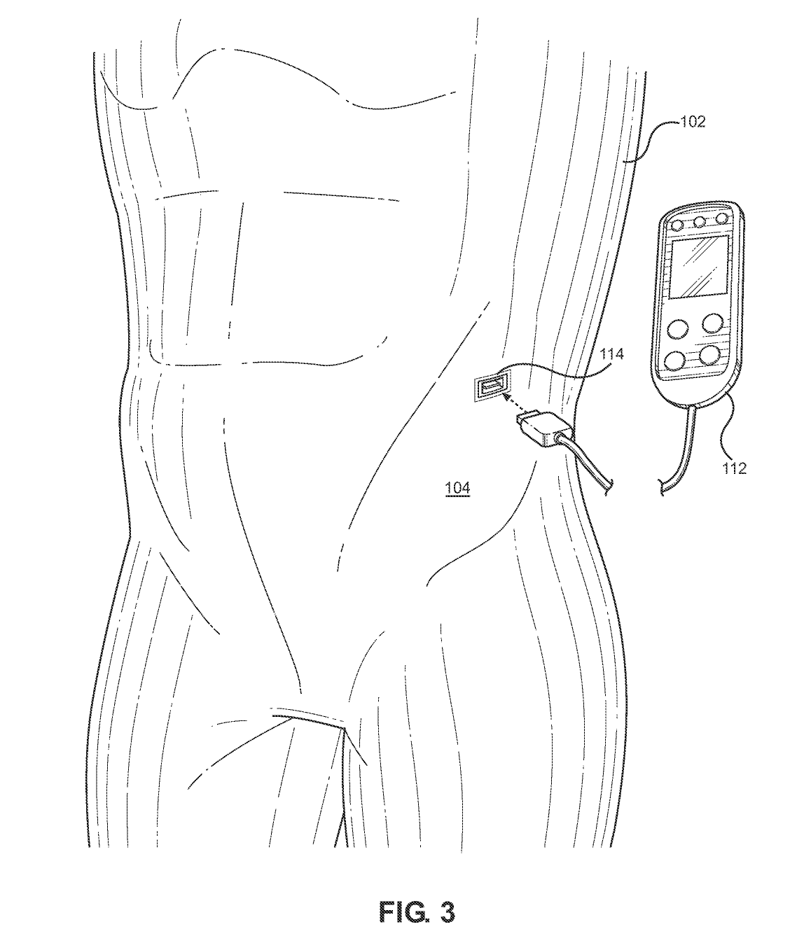

Referring now to FIG. 3, there is shown a close up view of the thermal body suit with a universal serial bus port according to one embodiment of the present invention. The unitary body suit 102 further includes a controller 112 adapted to control heat produced by the FIR coils. The controller 112 includes an input device to facilitate entry of a desired temperature. The input device may be a control panel on a touch screen. The controller 112 is also adapted to control the time of therapy, as well as other desired functions.

The unitary body suit 102 further includes a universal serial bus port 114 disposed on the outer layer 104 of the unitary body suit 102. The universal serial bus port 114 is in electrical communication with the FIR coils. Heat is provided to the unitary body suit 102 via the FIR coils which receive their energy from electrical potential delivered by the universal serial bus port 114 via an external power source. Some embodiments have a power source permanently integrated into the suit, others have a separable power source. The power source is preferably a battery pack, wherein the batteries can be disposable or rechargeable.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A thermal body suit that provides heat therapy, comprising:
a unitary body suit comprising an outer layer and an inner layer, wherein an insulation layer is disposed between the outer layer and the inner layer;
a plurality of FIR coils disposed in the insulation layer, the plurality of FIR coils sandwiched between the outer layer and the inner layer wherein said FIR coils are adapted to produce heat;
a controller adapted to control the plurality of FIR coils, wherein the controller includes an input device to facilitate entry of a desired temperature; and
a universal serial bus port disposed on the outer layer of the unitary body suit, wherein the universal serial bus port is in electrical communication with the FIR infrared coils.

2) The thermal body suit of claim 1, further comprising a fastener disposed on a side of the unitary body suit so as to allow a user to wear the body suit.

3) The thermal body suit of claim 2, wherein the fastener is a zipper.

4) The thermal body suit of claim 1, wherein the unitary body suit comprises a torso portion, a pair of sleeve portions, and a pair of leg portions.

5) The thermal body suit of claim 1, further comprising a power source in electrical communication with said plurality of FAR coils.

6) The thermal body suit of claim 1, wherein the controller is a remote control that sets a temperature and a time.

7) The thermal body suit of claim 1, further comprising a temperature sensor configured to measure temperature of a user and the unitary body suit.
